(19) United States
(12) Patent Application Publication
(10) Pub. No.: US 2022/0355051 A1
(43) Pub. Date: Nov. 10, 2022

(54) GENERATING SETTINGS FOR VENTILATORS USING MACHINE LEARNING TECHNIQUES

(71) Applicant: Verifai Inc., San Francisco, CA (US)

(72) Inventor: Sandeep Srinivasan, Palo Alto, CA (US)

(21) Appl. No.: 17/737,811

(22) Filed: May 5, 2022

Related U.S. Application Data
(60) Provisional application No. 63/184,367, filed on May 5, 2021.

(51) Int. Cl. A61M 16/00; A61M 16/01; G16H 20/40; G16H 40/67

(52) U.S. Cl. CPC A61M 16/024; A61M 16/01; G16H 20/40; G16H 40/67

(57) ABSTRACT

Methods, systems, and devices for generating optimal knob settings for ventilators using machine learning techniques are described. A system may collect a set of data associated with a target subject. The system may select a protocol based on the set of data, the protocol including settings associated with delivering therapy to the target subject via a device. The system may perform a control measure in response to selecting the protocol. Performing the control measure includes delivering the therapy to the target subject, via the device, based on the protocol. The set of data may include physiological information associated with the target subject or demographics information associated with the target subject.

### CROSS-REFERENCE TO RELATED APPLICATIONS

The present application claims the benefit of U.S. Provisional Application Ser. No. 63/184,367 filed May 5, 2021. The entire disclosure of the application listed is hereby incorporated by reference, in its entirety, for all that the disclosure teaches and for all purposes.

### FIELD OF THE INVENTION

The present invention is related generally to machine learning (ML), and specifically, reinforcement learning (RL) and imitation learning (IL) to control medical devices.

### BACKGROUND

Mechanical ventilators are devices that move air in and out of the lungs of a patient. Improved techniques associated with determining ventilator settings are desired.

### SUMMARY

The described techniques relate to improved methods, systems, devices, and apparatuses that support generating knob settings (e.g., optimal knob settings) for ventilators using machine learning (ML), and specifically reinforcement learning (RL) and imitation learning (IL), for faster weaning.

A system including: a processor; and a memory storing instructions thereon that, when executed by the processor, cause the processor to: collect a set of data associated with a target subject; select a protocol based on the set of data, the protocol including one or more settings associated with delivering therapy to the target subject via a device; and perform a control measure in response to selecting the protocol, wherein performing the control measure includes delivering the therapy to the target subject, via the device, based on the protocol.

Any of the aspects herein, wherein the set of data includes at least one of: physiological information associated with the target subject; and demographics information associated with the target subject; and selecting the protocol is based on at least one of the physiological information and the demographics information.

Any of the aspects herein, wherein: the set of data includes sedation information associated with the target subject, the sedation information including at least one of: one or more sedation settings associated with sedating the target subject, the one or more sedation settings including a sedation type and a sedation dosage; and a degree of consciousness of the target subject; and selecting the protocol is based on the sedation information.

Any of the aspects herein, wherein: the set of data includes intubation information associated with a set of intubations and the target subject, the intubation information including at least one of: a quantity of the set of intubations with respect to a temporal instance or a temporal period; and a temporal duration associated with an existing intubation of the set of intubations; and selecting the protocol is based on the intubation information.

Any of the aspects herein, wherein the instructions are further executable by the processor to: assign a classification to the target subject based on the set of data, wherein selecting the protocol is based on the classification.

Any of the aspects herein, wherein performing the control measure includes: transmitting the one or more settings to the device, a communication device associated with one or more personnel, or both.

Any of the aspects herein, wherein the instructions are further executable by the processor to: collect a second set of data associated with the target subject in response to performing the control measure; perform a second control measure in response to processing the second set of data, wherein performing the second control measure includes: adjusting or maintaining the one or more settings based on the second set of data; and delivering therapy to the target subject, via the device, based on adjusting or maintaining the one or more settings.

Any of the aspects herein, wherein the instructions are further executable by the processor to: compare at least a portion of the second set of data to a set of target criteria, the set of target criteria including at least one of: a target physiological parameter; and a target treatment outcome; and adjusting or maintaining the one or more settings based on a result of the comparing.

Any of the aspects herein, wherein: the protocol includes a baseline configuration associated with delivering the therapy to the target subject, wherein the baseline configuration includes: the one or more settings; and respective weighting factors corresponding to the one or more settings.

Any of the aspects herein, wherein: the device includes a ventilator; and the one or more settings include one or more device settings associated with the ventilator.

Any of the aspects herein, wherein the one or more settings include: a recommendation to intubate or extubate the target subject; and temporal information associated with the recommendation.

Any of the aspects herein, wherein the instructions are further executable by the processor to: provide at least a portion of the set of data to a machine learning model; and receive an output from the machine learning model in response to the machine learning model processing at least the portion of the set of data, the output including at least one of: an indication of a classification to the target subject; an indication of the protocol; and an indication of the one or more settings.

Any of the aspects herein, wherein processing at least the portion of the set of data by the machine learning model includes: generating predicted physiological information associated with the target subject based on at least the portion of the set of data; and comparing the predicted physiological information to target physiological information, wherein the output from the machine learning model is based on a result of the comparing.

Any of the aspects herein, wherein: the machine learning model performs one or more iterations of a control system loop, the control system loop including: providing the output; collecting an additional set of data, the additional data including physiological information associated with the target subject; generating additional predicted physiological information; comparing the additional predicted physiological information to the target physiological information; and providing an additional output, the additional output including one or more additional settings associated with delivering the therapy to the target subject via the device.

Any of the aspects herein, wherein the machine learning model is a software machine learning model.

Any of the aspects herein, wherein the instructions are further executable by the processor to: train and validate the machine learning model based on a comparison of the one or more settings to historical data associated with delivering the therapy to a set of subjects, wherein the historical data includes a set of previously applied settings associated with delivering the therapy to the set of subjects.

Any of the aspects herein, wherein the instructions are further executable by the processor to: train and validate the machine learning model based on a comparison of the one or more settings to a set of proposed settings associated with delivering the therapy to the target subject, wherein the set of proposed settings is included in data provided by personnel in association with delivering the therapy to the target subject.

A system including: a therapy device; a processor; and a memory storing instructions thereon that, when executed by the processor, cause the processor to: collect a set of data associated with a target subject; select a protocol based on the set of data, the protocol comprising one or more settings associated with delivering therapy to the target subject via the therapy device; and perform a control measure in response to selecting the protocol, wherein performing the control measure comprises delivering the therapy to the target subject, via the therapy device, based on the protocol.

Any of the aspects herein, wherein: the set of data comprises at least one of: physiological information associated with the target subject; and demographics information associated with the target subject; and selecting the protocol is based on at least one of the physiological information and the demographics information.

A method including: collecting a set of data associated with a target subject; selecting a protocol in response to collecting the set of data, the protocol comprising one or more settings associated with delivering therapy to the target subject via a device; and performing a control measure in response to selecting the protocol, wherein performing the control measure comprises delivering the therapy to the target subject, via the device, based on the protocol.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/implementations in combination with any one or more other aspects/features/implementations.

Use of any one or more of the aspects or features as disclosed herein.

It is to be appreciated that any feature described herein can be claimed in combination with any other feature(s) as described herein, regardless of whether the features come from the same described implementation.

The details of one or more aspects of the disclosure are set forth in the accompanying drawings and the description below. Other features, objects, and advantages of the techniques described in this disclosure will be apparent from the description and drawings, and from the claims.

The preceding is a simplified summary of the disclosure to provide an understanding of some aspects of the disclosure. This summary is neither an extensive nor exhaustive overview of the disclosure and its various aspects, implementations, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, implementations, and configurations of the disclosure are possible utilizing, alone or in combination, one or more of the features set forth above or described in detail below.

Numerous additional features and advantages of the present disclosure will become apparent to those skilled in the art upon consideration of the implementation descriptions provided hereinbelow.

### BRIEF DESCRIPTION OF THE DRAWINGS

FIG. 1 illustrates an example of a system in accordance with aspects of the present disclosure.

FIG. 2 illustrates an example of a user interface in accordance with aspects of the present disclosure.

FIG. 3 illustrates an example of a system in accordance with aspects of the present disclosure.

FIG. 4 illustrates an example of a process flow in accordance with aspects of the present disclosure.

FIG. 5 illustrates an enlarged view of example device settings of a ventilator supported by aspects of the present disclosure.

### DETAILED DESCRIPTION

Generating the optimal knob-settings for a ventilator used in an Intensive Care Unit (“ICU”), critical care units, and nursing facilities can be important. As the number of patients increase in an ICU, the biggest challenge with respiratory infections like COVID is to wean patients off the ventilator quickly, so they can breathe on their own. The longer it takes to wean, the higher the risk of developing ventilator dependence, i.e. the patient's brain forgets how to breathe, perhaps due to interference from the prefrontal cortex (fear, anxiety etc.)

This is of significance because the delay in outflow of patients from the ICU to lower level critical care can significantly limit inflow of patients in need.

Simply making more ventilators may not solve the problem, since there are a limited number of trained RTs and physicians who know how to manage ventilator settings and weaning protocols. Also, there are not enough RTs to take care of more than 100 k ICU patients.

The weaning methods used by RTs (respiratory therapists) and Critical Care doctors, are at best ‘ad-hock’, leading to prolonged weaning schedules and ventilator dependence for the patient.

In critical times, such as now, we need to come up with dynamic ventilator weaning protocols. One such opportunity is to use ML, and specifically RL/IL, to minimize ventilator use and optimize weaning RL/IL recommended knob-settings for the ventilator can be used to assist the RT or ICU doctors to make better decisions, which would typically be non-obvious. A combination of Reinforcement Learning and Imitation Learning can lead to significant reduction in weaning patients off ventilators.

The extubation rates in New York for COVID-19 patients was reported to be close to 50%, while it was reported to be 20% in China. These are alarmingly low rates. Even the most skilled physician or respiratory therapist (RT) can make sub optimal decisions that lead to negative outcomes (e.g., reintubation), in particular during times of crisis, where the critical care-patient to doctor (or RT) ratio is very high.

Aspects of the present disclosure support a machine learning based application that learns and recommends optimal ventilator settings as a function of time, with an objective to optimize successful extubation. Such an application will assist physicians and RTs significantly in a pandemic such as COVID-19 and beyond.

Aspects of the present disclosure utilize a combination of Reinforcement Learning (RL) and Imitation Learning (TL) that continuously learns by imitating experts and dynamically adapts to changing conditions. The goal is to achieve super human learning capability to react to subtle patient observables that surpass the capabilities of even the most experienced physicians, in order to maximize extubation success.

Aspects described herein may provide assistance to RTs and critical care physicians worldwide with the combined experiences of millions of experts and billions of hours of observations. Such data (e.g., experiences, observations, etc.) are captured in a machine learning model.

### Example Overview

Making decisions for weaning ICU patients from invasive mechanical ventilation and regulating their associated analgesia during ventilation represents a difficult problem which currently is not being systematically addressed in other systems. Various existing protocols differ in their recommendations (e.g., treatment recommendations, ventilator settings, etc.), often resulting in ad-hoc decisions made even by attending physicians. Moreover, these attending physicians usually only see these patients once or twice daily to make adjustments to the multiple settings of the ventilators, often incorporating trial and error. To make matters even worse, the remaining hourly adjustments to the ventilator settings are made by respiratory technicians who have far less training.

The concerns are that prolonged dependence on mechanical ventilation and premature extubating of the patient result in increasing the risk of complications. Weaning the patient from the ventilator treatment too late can increase the risks of ventilator dependency, post-extubation delirium, drug dependence, length of stay in the ICU, and ventilator-induced pneumonia (VAP). Weaning too early risks reintubation, not only leading to longer hospital stays but also to a five-fold higher risk of VAP and other complications, resulting in a seven-fold higher risk of death.

In addition to the lack of expert consensus or clear guidelines, there are other complications related to the ICU recorded data including: unobservable variables such as muscle weakness resulting in compromised airways, sparsity and noisiness of recorded data, large numbers of potential combinations of sedation types/dosages and ventilator knob settings (i.e. the decision space is very large), and granularity in the observation times of events (e.g., such as for successful and unsuccessful extubations) compared to the actual times of extubation readiness and uncertainties in how premature an unsuccessful extubation actually was.

Aspects of the present disclosure provide a principled decision support tool using ongoing measurements of vital signs, current and past medication dosages, and current and past ventilator settings to aid ICU physicians and technicians to predict when it would be best to extubate, as well as the amounts and types of sedations, and adjustments to the ventilator settings as a function of time.

The systems described herein support a real-time, software-only, decision support tool which is ventilator and vendor agnostic. This tool will incorporate deep reinforcement learning (Deep RL) and imitation learning (IL) for incorporating certain aspects of existing expert protocols. Moreover, it will also incorporate various algorithms to drastically shorten the training time for the machine learning algorithms.

Previous studies have identified anywhere from 32 to 43 variables related to the patient's state which are relevant to the intubation weaning problem. These variables include, but are not limited to: patient demographics (e.g., age, weight, gender, admit type, ethnicity), patient physiological information (e.g., fluid status, blood pressure, central venous pressure, etc.), ventilator settings, time of ventilation & number of intubations up to present time, level of consciousness (from the Richmond Agitation Sedation Scale (RASS)), and current dosages.

The RL action space as a function of time may include the ventilator knob settings, optionally along with sedation-specifics such as type and dosage. The RL rewards consist of observable encouraging vital signs and successful extubations, as well as penalties for adverse vital signs and unsuccessful extubations (e.g., extubations resulting in later reintubation). Aspects of the present disclosure support achieving an RL/IL capability to react to subtle patient observables that surpass the capabilities of even experienced physicians in order to maximize long term reward and extubation success.

Aspects of the present disclosure provide improvements for addressing a critical need to provide scalable ventilator management application for ICU physicians and respiratory therapists (RT). A study done by the Innova Fairfax Hospital states that 42% of Covid-19 patients that were on invasive mechanical ventilation (IMV) died. While this is an alarming rate, this report also suggests this rate is much lower than in other facilities across the country and the world. The number of patients on ventilators during COVID was alarming high, and their mortality rate was greater than 40%. COVID-19 has exposed the fragility of health care systems worldwide, including equipment such as ventilators, and medical resources such as trained doctors and respiratory therapists (RTs). There are an estimated 62,000 functioning ventilators in the US.

Medical errors are a leading cause of death. A study from Harvard University found that one of the 8 leading causes of deaths in the United States is due to medical errors. In some cases, a disproportionate ICU doctor-to-patient ratio compounds the problem.

The alarming second wave in India has filled up ICU beds in all major cities. The hospitals are operating at 99% occupancy, with limited ventilator resources and trained physicians to monitor and adjust the ventilator settings. All these factors play into the high mortality rates for COVID-19 patients on IMV.

A scalable remote ventilator management system described herein by the present disclosure will increase the number of patients a single physician can manage, without an increase in medical errors. In some aspects, the system may support remote management of ventilators. For example, a nurse practitioner or an RT could install ventilators in people's homes, and an expert ICU physician can manage these home patients remotely.

Aspects of the systems described herein support dynamic ventilator settings management by ICU physicians and respiratory therapists to manage a large number of patients safely and remotely, in times of COVID-19 and in a non-pandemic future. For example, Initial reports from China found mortality rates of 90% among COVID-19 patients with ARDS (adult respiratory distress syndrome) on IMV. New York hospitals showed mortality rate of 88%. While these are alarming rates, the reality is somewhat lower, but still very high. The COVID-19 pandemic has shown that ventilator settings play a critical role in outcomes for patients with ARDS.

Aspects of the present disclosure systematically address problems associated with making decisions for weaning ARDS patients from invasive mechanical ventilation and regulating their associated analgesia during ventilation. Various existing protocols differ in their recommendations, often resulting in ad-hoc decisions made even by experienced attending physicians. Moreover, these attending physicians usually only see these patients once or twice daily to make adjustments to the multiple settings of the ventilators, often incorporating trial and error. Hourly adjustments to the ventilator settings are uncommon and are typically made by respiratory therapists who have lesser training than an attending physician.

The systems described herein support addressing concerns such as increased risk of complications due to prolonged dependence on mechanical ventilation and premature extubating of a patient. Weaning too late can increase the risks of ventilator dependency, post-extubation delirium, drug dependence, length of stay in the ICU, and ventilator-induced pneumonia (VAP). Weaning too early risks reintubation, not only leading to longer hospital stays but also to a five-fold higher risk of VAP and other complications, resulting in a seven-fold higher risk of death. There were 200,00 to 300,00 cases of ventilator related VAP in the USA in 2019.

Aspects of the present disclosure provide positive impacts related to medical costs. Faster weaning off the ventilators will result in significant cost savings for healthcare systems, and improves ICUs throughput, thereby generating higher revenue due to an increased number of patients a facility can admit.

Example statistics include: US ICUs that require ventilation every year (Pre covid) >2.5 million. As of May 2020, 4.7 Million patients are suffering due COVID 19. Average Cost of ICU Patient on ventilator=$2300 per day. After the fourth day, costs rise to $3900 per day. Even a 20% reduction in days spent on ventilators can result in significant improvement in ICU throughput and hospital revenue increase. Potential cost savings of $10 billion in the US, for healthcare providers, insurance companies, and significant opportunities for revenue growth. Global ventilator market size was $2.5 Billion in 2019 and is projected to reach $9.13 Billion by 2027.

A system described herein support an AI application that learns from millions of prior successful protocols and real-time data from the patients observables. The AI application may recommend ventilator settings for a large number of patients simultaneously. Such a dynamic Al based system will increase successful extubation, reduce reintubations and accelerate weaning by a significant margin based on learning from millions of priors and learning in real-time from the patients' vital, blood chemistry and other observables. The system can assist attending physicians and respiratory therapists (RTs) via an Al mobile application to dynamically recommend real-time remote control of the ventilator settings for multiple patients remotely. Even in non-pandemic settings, the typical ratio of attending physicians to patients can be greater than 1:10 to 1:30. Even in a non-pandemic settings, periodic adjustments to the ventilators are typically postponed to coincide with the doctors availability, although the patient could benefit from more frequent ventilator adjustments.

The system described herein may assist medical staff with an Al mechanism to dynamically recommend real-time remote control of the ventilator settings. Aspects of the Al mechanism may provide recommendations based on complications related to the ICU recorded data including, but not limited to: unobservable variables such as muscle weakness resulting in compromised airways, sparsity and noisiness of recorded data, large numbers of potential combinations of sedation types/dosages and ventilator knob settings (i.e., the decision space for even the best human physicals and respiratory experts is very large). Using the Al mechanism described herein, the system may apply the learnings from millions of prior outcomes coupled with real-time patient data and make significantly better decisions than an expert human (e.g., an expert attending physician).

Protocoled Ventilator Weaning Results in Improved Outcomes

Recent studies by researchers, clinicians and device companies have shown that using protocoled ventilator weaning results in improved outcomes such as the following: ICU length of stay decreased by 11%, mean duration of ventilation decreased by 26%, average savings in total ICU costs in the range of $3,000 to $5,000 (per case), reduced mortality rate of 7%, reduction in rates of unsuccessful extubations in the range of 6.3% to 9.7%, reduced weaning duration by 70%, and decrease in VAP by 9%. The conclusion from these recent studies is that protocol based ventilator weaning can provide measurable improved results for patients with corresponding reductions in ICU costs. While protocoled ventilator weaning shows a positive trajectory, in some systems, there is a lack of expert consensus or clear guidelines for ventilator protocols to use per disease or condition.

Aspects of the present disclosure (e.g., a ventilator management application) provide a medical impact that is significant during pandemics and non-pandemic situations. For instance, in large cities in India and China, there are a significant number of patients on the ventilator each winter (e.g., due to high levels of PM 2.5 particles due to air pollution). The number of ICU beds required in the winter months in India and China are disproportionally high, leading to stress on the medical system each year.

According to example aspects of the present disclosure, a real-time, Deep Reinforcement Learning (DRL) based decision support software agent is described. The software agent (also referred to herein as a DRL software agent) is ventilator and vendor agnostic. The software agent uses online and offline DRL to learn from existing expert protocols and anonymized historical outcomes. An offline DRL agent learns a policy from an existing dataset, which it will later use in deployment. Online DRL has an environment for the software agent to interact with to learn a good policy (e.g., a policy which, when implemented, may achieve a target outcome). The online DRL agent interacts with the environment (e.g., ventilator settings and the patient) in real-time and learns a policy (e.g., a dynamic online policy) of knob-settings for the ventilator. By combining the dynamic online policy with previously learnt offline policies (i.e., agents learnings from offline DRL), the DRL software agent is able to achieve an optimal policy for the current time interval.

The DRL agent is modeled as a Markov decision process (MDP), where, given a dataset of samples (also referred to herein “priors”) of ventilator knob-settings and corresponding patient outcomes, Al algorithms may learn a policy that is superior than the best policies in the dataset. For example, using a combination of offline and online DRL, the DRL software agent can learn a policy that will generate superior knob-settings for a current patient in comparison to previously learnt policies (or protocols).

Using a combination of offline and online DRL algorithms, the DRL software agent can learn a new policy that will generate superior ventilator knob-settings for the patient, in comparison to previously learnt protocols and expert physician settings. Combining offline and online DRL algorithms, as supported by the present disclosure, may drastically shorten the training time for training the DRL models to learn an optimal policy. Aspects of the present disclosure include training and/or implementing the DRL models based on 32 to 43 variables related to the patient's state which are relevant to the extubation weaning problem. The variables include, but are not limited to: patient demographics (e.g., age, weight, gender, admit type, ethnicity, etc.), patient physiological information (e.g., arterial and venous blood gasses, fluid status, blood pressure, central venous pressure etc.), ventilator knob settings (e.g., assist control volume (AC), tidal volume (Vt), flow rate (IFR), ventilation rate (RR), FiO2, PEEP, etc.), time (e.g., temporal instance or duration) of ventilation and number of intubations up to a present time, level of consciousness (e.g., from the Richmond Agitation Sedation Scale (RASS)), and current dosages.

The DRL action space as a function of time includes ventilator knob settings, and in some optional implementations, sedation-specifics such as type and dosage. Rewards may include observable encouraging vital signs and successful extubations, as well as penalties for adverse vital signs and unsuccessful extubations. Aspects of the present disclosure provide a system capable of achieving a capability (policy) to react to subtle patient observables that surpass the capabilities of even experienced physicians in order to maximize long term reward (e.g., faster weaning, extubation success, and reduced rates of reintubation).

Aspects of the present disclosure include optimization and validation of offline and online DRL agents. In an example, a first aim (also referred to herein “Aim 1”) is to optimize and validate an offline DRL agent that recommends ventilator knob settings, and further, to compare the recommended ventilator knob settings to existing public datasets as in clinical outcomes for ICU patients. The dataset is called the Multi-parameter Intelligent Monitoring in Intensive Care (MIMIC III) database. The MIMIC III dataset contains anonymized critical care data for 50 k adult admissions, and from this dataset, Aim 1 includes using the 8000+ adults that were under ventilation. The aim would be to show that the offline DRL learnings from this dataset would lead to a better policy for a set of patients (e.g., 1000 patients or more). An example of better policy is defined as ventilator settings that would lead to extubations within 24 hours, but is not limited thereto.

In another example, a second aim (also referred to herein as “Aim 2”) is to optimize and validate an online DRL agent that has previously learned a policy from the MIMIC III dataset. The online DRL agent will recommend knob and/or sedation settings for an ICU patient, Aim 2 includes comparing the recommended knob settings to ventilator and/or sedation settings used by an attending physician in an ICU setting. In an example, a successful result includes when (1) the proposed approaches described herein (of using offline DRL to train a software agent that recommends ventilator knob settings performs better than existing protocols in a given dataset) (2) will lead to a synthesis of the most effective parts of the multiple existing protocols, resulting in a more effective hybrid protocol for managing and controlling ventilators. Other example factors associated with measuring success of the proposed approaches include reduced mortality rate, reduced weaning durations, and reduced VAP and reintubation rates for ARDS patients on invasive ventilators.

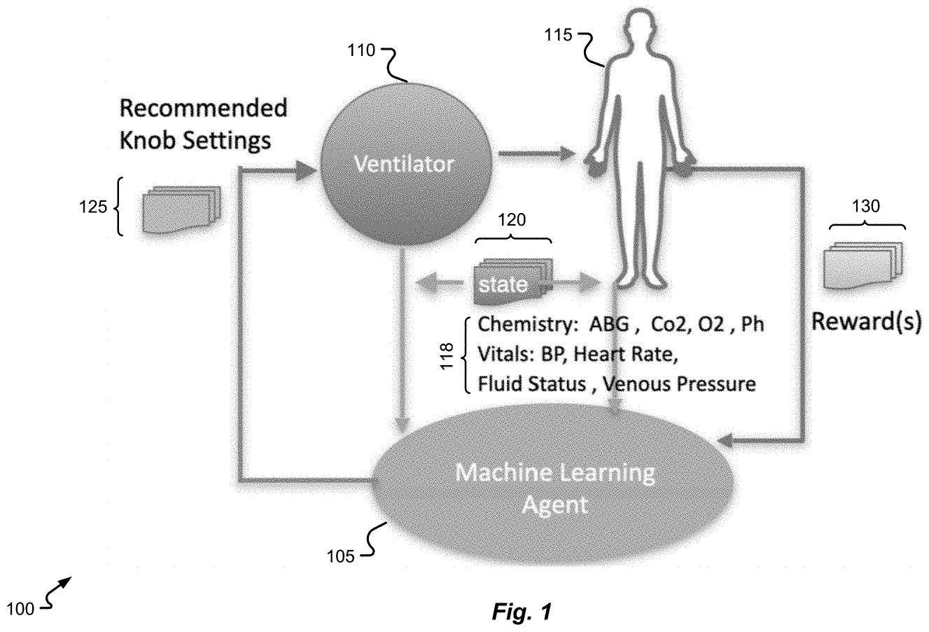

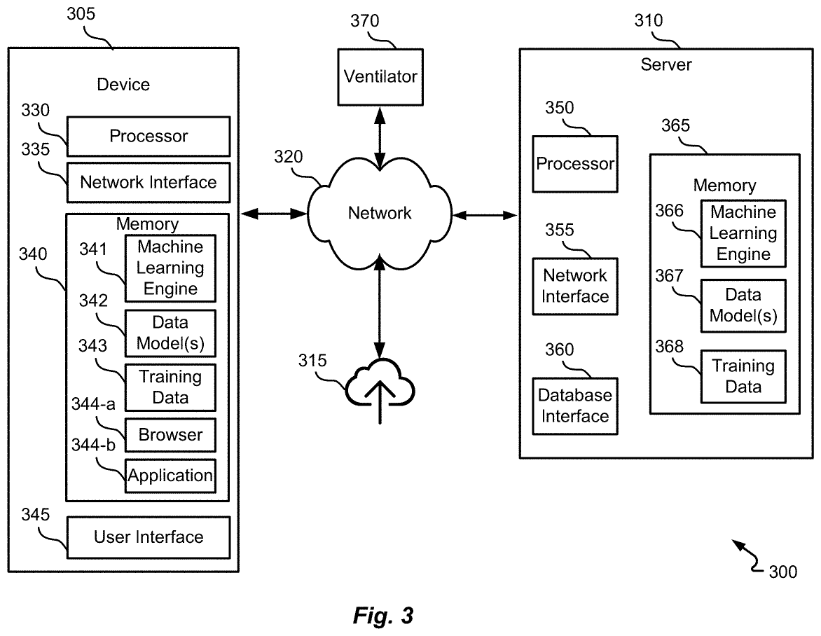

FIG. 1 illustrates an example of a system 100 that supports generating optimal knob settings for ventilators using machine learning techniques in accordance with aspects of the present disclosure. The system 100 may include a machine learning agent 105 (e.g., a DRL software agent described herein) and a ventilator 110 (also referred to herein as a ventilator device). Example aspects of the system 100 may be implemented by a system 300 later described with reference to FIG. 3. For example, the machine learning agent 105 may be implemented by a device 305 and/or a machine learning engine 341 of FIG. 3.

Referring to FIG. 1, the machine learning agent 105 may collect data 118 associated with a target subject 115. The data 118 may include physiological information (e.g., chemistry: ABG, CO2, O2, PH; vitals: blood pressure, heart rate; fluid status; venous pressure; etc.) associated and/or demographics information associated with the target subject 115 as described herein. In some aspects, the machine learning agent 105 may collect state information 120 associated with the target subject 115 and the physiological information. The machine learning agent 105 may collect data 118 and/or state information 120 from the ventilator 110 or sensor devices (not illustrated).

The machine learning agent 105 may generate and provide knob settings 125 (e.g., recommended knob settings) to the ventilator 110 in association with delivering therapy to the target subject 115 via the ventilator 110. The machine learning agent 105 may provide the knob settings 125 to a device associated with medical personnel (e.g., a respiratory technician). The machine learning agent 105 may generate the knob settings 125 in association with achieving one or more rewards 130.

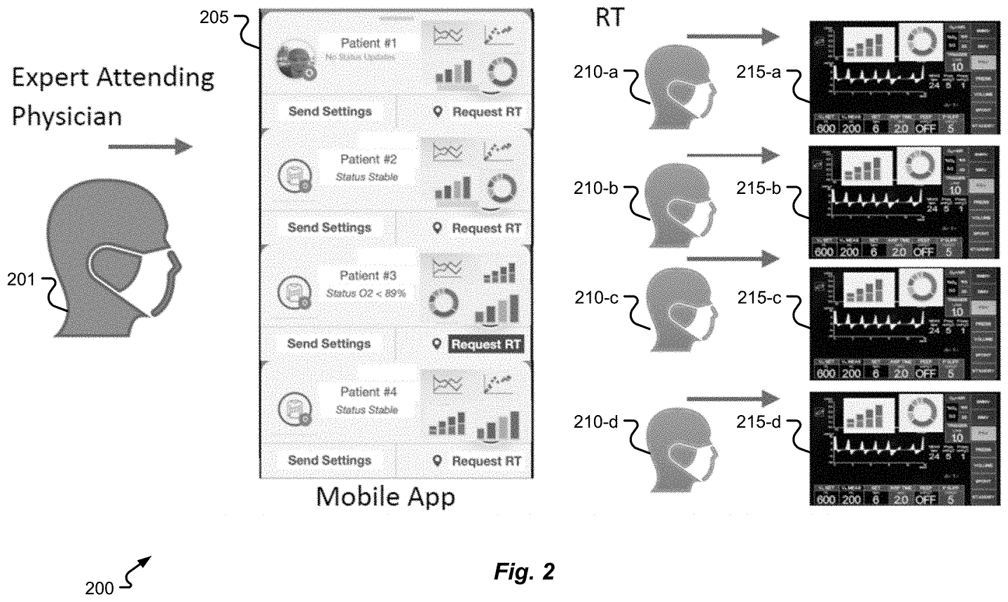

FIG. 2 illustrates an example 200 that supports aspects of the present disclosure.

Referring to FIG. 2, aspects of the present disclosure support an application 205 (e.g., a mobile application, a desktop application, etc.) via which a user 201 may manage a relatively large number of ventilator patients (e.g., 100 or more patients) simultaneously. The user 201 may be, for example, medical personnel (e.g., an attending physician). Via the application 205, the user 201 may transmit data including device settings 215 (e.g., knob settings, etc.) to a ventilator. Accordingly, for example, the user 201 may control the device settings 215 of the ventilator via the application 205. Additionally, or alternatively, via the application 205, the user 201 may transmit a request to a device of other medical personnel 210 (e.g., a respiratory technician). The request may include instructions to the medical personnel to apply the device settings 215 to the ventilator (e.g., adjust knob settings of the ventilator).

Aspects of FIG. 2 may be implemented by the system 300 later described with reference to FIG. 3. For example, aspects of FIG. 2 may be implemented by a device 305, an application 344, and/or a user interface 345 of FIG. 3.

FIG. 3 illustrates an example of a system 300 that supports generating optimal knob settings for ventilators using machine learning techniques in accordance with aspects of the present disclosure. In some examples, the system 300 may be implemented by aspects of the system 100 described with reference to FIG. 1. The system 300 may include a device 305, a server 310, a database 315, a communication network 320, and a ventilator 370. The devices 305, the server 310, the database 315, the communications network 320, and the ventilator 370 may implement aspects of the present disclosure described herein.

In various aspects, settings of the any of the device 305, the server 110, database 315, and the network 320 may be configured and modified by any user and/or administrator of the system 300. Settings may include thresholds or parameters described herein, as well as settings related to how data is managed. Settings may be configured to be personalized for one or more devices 305, users of the devices 305, and/or other groups of entities, and may be referred to herein as profile settings, user settings, or organization settings. In some aspects, rules and settings may be used in addition to, or instead of, parameters or thresholds described herein. In some examples, the rules and/or settings may be personalized by a user and/or administrator for any variable, threshold, user (user profile), device 305, entity (e.g., patient), or groups thereof.

A device 305 may include a processor 330, a network interface 335, a memory 340, and a user interface 345. In some examples, components of the device 305 (e.g., processor 330, network interface 335, memory 340, user interface 345) may communicate over a system bus (e.g., control busses, address busses, data busses) included in the device 305. In some cases, the device 305 may be referred to as a computing resource.

In some cases, the device 305 may transmit or receive packets to one or more other devices (e.g., another device 305, the server 310, the database 315, the ventilator 370) via the communication network 320, using the network interface 335. The network interface 335 may include, for example, any combination of network interface cards (NICs), network ports, associated drivers, or the like. Communications between components (e.g., processor 330, memory 340) of the device 305 and one or more other devices (e.g., another device 305, the database 315) connected to the communication network 320 may, for example, flow through the network interface 335.

The processor 330 may correspond to one or many computer processing devices. For example, the processor 330 may include a silicon chip, such as a FPGA, an ASIC, any other type of IC chip, a collection of IC chips, or the like. In some aspects, the processors may include a microprocessor, CPU, a GPU, or plurality of microprocessors configured to execute the instructions sets stored in a corresponding memory (e.g., memory 340 of the device 305). For example, upon executing the instruction sets stored in memory 340, the processor 330 may enable or perform one or more functions of the device 305.

The processor 330 may utilize data stored in the memory 340 as a neural network (also referred to herein as a machine learning network). The neural network may include a machine learning architecture. In some aspects, the neural network may be or include an artificial neural network (ANN). In some other aspects, the neural network may be or include any machine learning network such as, for example, a deep learning network, a convolutional neural network, or the like. Some elements stored in memory 340 may be described as or referred to as instructions or instruction sets, and some functions of the device 305 may be implemented using machine learning techniques.

The memory 340 may include one or multiple computer memory devices. The memory 340 may include, for example, Random Access Memory (RAM) devices, Read Only Memory (ROM) devices, flash memory devices, magnetic disk storage media, optical storage media, solid-state storage devices, core memory, buffer memory devices, combinations thereof, and the like. The memory 340, in some examples, may correspond to a computer-readable storage media. In some aspects, the memory 340 may be internal or external to the device 305.

The memory 340 may be configured to store instruction sets, neural networks, and other data structures (e.g., depicted herein) in addition to temporarily storing data for the processor 330 to execute various types of routines or functions. For example, the memory 340 may be configured to store program instructions (instruction sets) that are executable by the processor 330 and provide functionality of machine learning engine 341 described herein. The memory 340 may also be configured to store data or information that is useable or capable of being called by the instructions stored in memory 340. One example of data that may be stored in memory 340 for use by components thereof is a data model(s) 342 (also referred to herein as a neural network model) and/or training data 343 (also referred to herein as a training data and feedback).

The machine learning engine 341 may include a single or multiple engines. The device 305 (e.g., the machine learning engine 341) may utilize one or more data models 342 for recognizing and processing information obtained from other devices 305, the server 310, the database 315, and the ventilator 370. In some aspects, the device 305 (e.g., the machine learning engine 341) may update one or more data models 342 based on learned information included in the training data 343. In some aspects, the machine learning engine 341 and the data models 342 may support forward learning based on the training data 343. The machine learning engine 341 and data models 342 may support reinforcement learning and imitation learning described herein. The machine learning engine 341 may have access to and use one or more data models 342. For example, the data model(s) 342 may be built and updated by the machine learning engine 341 based on the training data 343. The data model(s) 342 may be provided in any number of formats or forms. Non-limiting examples of the data model(s) 342 include Decision Trees, Support Vector Machines (SVMs), Nearest Neighbor, and/or Bayesian classifiers.

The machine learning engine 341 may create, select, and execute processing decisions as described herein. Processing decisions may be handled automatically by the machine learning engine 305, with or without human input.

The machine learning engine 341 may store, in the memory 340 (e.g., in a database included in the memory 340), historical information. Data within the database of the memory 340 may be updated, revised, edited, or deleted by the machine learning engine 341. In some aspects, the machine learning engine 341 may support continuous, periodic, and/or batch fetching of data and data aggregation.

The device 305 may render a presentation (e.g., visually, audibly, using haptic feedback, etc.) of an application 344 (e.g., a browser application 344-a, an application 344-b). The application 344-b may be an application associated with executing, controlling, and/or monitoring the ventilator 370 described herein. For example, the application 344-b may enable control of the device 305 or the ventilator 370.

In an example, the device 305 may render the presentation via the user interface 345. The user interface 345 may include, for example, a display (e.g., a touchscreen display), an audio output device (e.g., a speaker, a headphone connector), or any combination thereof. In some aspects, the applications 344 may be stored on the memory 340. In some cases, the applications 344 may include cloud-based applications or server-based applications (e.g., supported and/or hosted by the database 315 or the server 310). Settings of the user interface 345 may be partially or entirely customizable and may be managed by one or more users, by automatic processing, and/or by artificial intelligence.

In an example, any of the applications 344 (e.g., browser application 344-a, application 344-b) may be configured to receive data in an electronic format and present content of data via the user interface 345. For example, the applications 344 may receive data from another device 305, the server 310, or the ventilator 370 via the communications network 320, and the device 305 may display the content via the user interface 345.

The database 315 may include a relational database, a centralized database, a distributed database, an operational database, a hierarchical database, a network database, an object-oriented database, a graph database, a NoSQL (non-relational) database, etc. In some aspects, the database 315 may store and provide access to, for example, any of the stored data described herein.

The server 310 may include a processor 350, a network interface 355, database interface instructions 360, and a memory 365. In some examples, components of the server 310 (e.g., processor 350, network interface 355, database interface 360, memory 365) may communicate over a system bus (e.g., control busses, address busses, data busses) included in the server 310. The processor 350, network interface 355, and memory 365 of the server 310 may include examples of aspects of the processor 330, network interface 335, and memory 340 of the device 305 described herein.

For example, the processor 350 may be configured to execute instruction sets stored in memory 365, upon which the processor 350 may enable or perform one or more functions of the server 310. In some aspects, the processor 350 may utilize data stored in the memory 365 as a neural network. In some examples, the server 310 may transmit or receive packets to one or more other devices (e.g., a device 305, the database 315, another server 310) via the communication network 320, using the network interface 355. Communications between components (e.g., processor 350, memory 365) of the server 310 and one or more other devices (e.g., a device 305, the database 315, the ventilator 370, etc.) connected to the communication network 320 may, for example, flow through the network interface 355.

In some examples, the database interface instructions 360 (also referred to herein as database interface 360), when executed by the processor 350, may enable the server 310 to send data to and receive data from the database 315. For example, the database interface instructions 360, when executed by the processor 350, may enable the server 310 to generate database queries, provide one or more interfaces for system administrators to define database queries, transmit database queries to one or more databases (e.g., database 315), receive responses to database queries, access data associated with the database queries, and format responses received from the databases for processing by other components of the server 310.

The memory 365 may be configured to store instruction sets, neural networks, and other data structures (e.g., depicted herein) in addition to temporarily storing data for the processor 350 to execute various types of routines or functions. For example, the memory 365 may be configured to store program instructions (instruction sets) that are executable by the processor 350 and provide functionality of the machine learning engine 366 described herein. One example of data that may be stored in memory 365 for use by components thereof is a data model(s) 363 (also referred to herein as a neural network model) and/or training data 368. The data model(s) 363 and the training data 368 may include examples of aspects of the data model(s) 342 and the training data 343 described with reference to the device 305. For example, the server 310 (e.g., the machine learning engine 366) may utilize one or more data models 363 for recognizing and processing information obtained from devices 305, another server 310, the database 315, or the ventilator 370. In some aspects, the server 310 (e.g., the machine learning engine 366) may update one or more data models 363 based on learned information included in the training data 368.

In some aspects, components of the machine learning engine 366 may be provided in a separate machine learning engine in communication with the server 310.

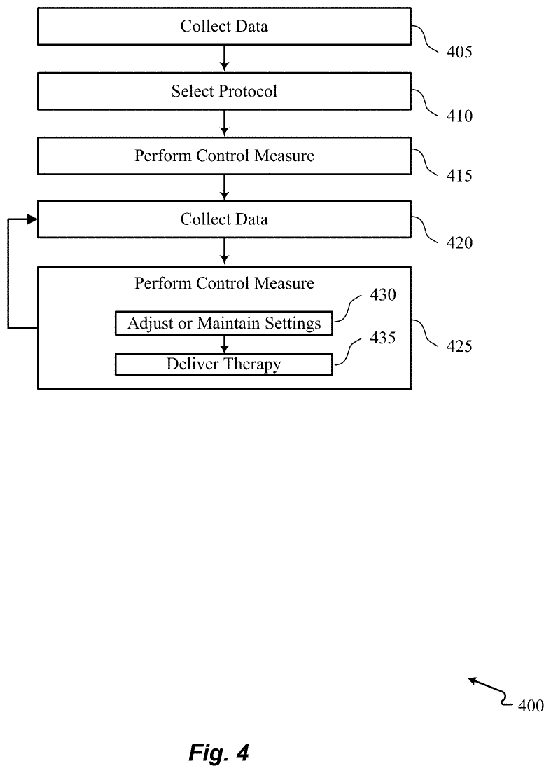

FIG. 4 illustrates an example of a process flow 400 that supports generating optimal knob settings for ventilators using machine learning techniques in accordance with aspects of the present disclosure. In some examples, process flow 400 may implement aspects of the system 100, example 200, and the system 300 described with reference to FIGS. 1 through 3.

In the following description of the process flow 400, the operations may be performed in a different order than the order shown, or the operations may be performed in different orders or at different times. Certain operations may also be left out of the process flow 400, or other operations may be added to the process flow 400. Operations of the process flow 400 may be performed autonomously (e.g., by a device 105) and/or semi-autonomously (based on a user input).

It is to be understood that while a device 105 is described as performing a number of the operations of process flow 400, any device (e.g., another device 105 in communication with the device 105) may perform the operations shown.

At 405, the process flow 400 may include collecting a set of data associated with a target subject;

At 410, the process flow 400 includes selecting a protocol based on the set of data, the protocol including one or more settings associated with delivering therapy to the target subject via a device.

In some aspects, the set of data may include at least one of: physiological information associated with the target subject; and demographics information associated with the target subject. In some aspects, selecting the protocol is based on at least one of the physiological information and the demographics information.

In some aspects, the set of data may include sedation information associated with the target subject, the sedation information including at least one of: one or more sedation settings associated with sedating the target subject, the one or more sedation settings including a sedation type and a sedation dosage; and a degree of consciousness of the target subject. In some aspects, selecting the protocol is based on the sedation information.

In some aspects, the set of data may include intubation information associated with a set of intubations and the target subject, the intubation information including at least one of: a quantity of the set of intubations with respect to a temporal instance or a temporal period; and a temporal duration associated with an existing intubation of the set of intubations. In some aspects, selecting the protocol is based on the intubation information.

In some aspects, the instructions are further executable by the processor to: assign a classification to the target subject based on the set of data. In some aspects, selecting the protocol is based on the classification.

At 415, the process flow 400 includes performing a control measure in response to selecting the protocol. In some aspects, performing the control measure may include delivering the therapy to the target subject, via the device, based on the protocol.

In some aspects, performing the control measure may include: transmitting the one or more settings to the device, a communication device associated with one or more personnel, or both.

At 420, the process flow 400 includes collecting a second set of data associated with the target subject in response to performing the control measure.

At 425, the process flow 400 includes perform a second control measure in response to processing the second set of data. In some aspects, performing the second control measure may include: adjusting or maintaining the one or more settings (at 430) based on the second set of data; and delivering therapy to the target subject (at 435), via the device, based on adjusting or maintaining the one or more settings.

In an example, the process flow 400 includes comparing at least a portion of the second set of data to a set of target criteria, the set of target criteria including at least one of: a target physiological parameter; and a target treatment outcome. In an example, adjusting or maintaining the one or more settings (at 430) is based on a result of the comparing.

In some aspects, the protocol may include a baseline configuration associated with delivering the therapy to the target subject. In some aspects, the baseline configuration may include: the one or more settings; and respective weighting factors corresponding to the one or more settings.

In some aspects, the device may include a ventilator; and the one or more settings may include one or more device settings associated with the ventilator.

In some aspects, the one or more settings may include: a recommendation to intubate or extubate the target subject; and temporal information associated with the recommendation.

In some aspects, the process flow 400 includes providing at least a portion of the set of data to a machine learning model, and the process flow 400 may include receiving an output from the machine learning model in response to the machine learning model processing at least the portion of the set of data, the output including at least one of: an indication of a classification to the target subject; an indication of the protocol; and an indication of the one or more settings.

In some aspects, processing at least the portion of the set of data by the machine learning model may include: generating predicted physiological information associated with the target subject based on at least the portion of the set of data; and comparing the predicted physiological information to target physiological information. In some aspects, the output from the machine learning model is based on a result of the comparing.

In some aspects, the machine learning model performs one or more iterations of a control system loop, the control system loop including: providing the output; collecting an additional set of data, the additional data including physiological information associated with the target subject; generating additional predicted physiological information; comparing the additional predicted physiological information to the target physiological information; and providing an additional output, the additional output including one or more additional settings associated with delivering the therapy to the target subject via the device.

In some aspects, the machine learning model is a software machine learning model.

In some aspects, the process flow 400 includes training and validating the machine learning model based on a comparison of the one or more settings to historical data associated with delivering the therapy to a set of subjects. In some aspects, the historical data may include a set of previously applied settings associated with delivering the therapy to the set of subjects.

In some aspects, the process flow 400 includes training and validating the machine learning model based on a comparison of the one or more settings to a set of proposed settings associated with delivering the therapy to the target subject. In some aspects, the set of proposed settings is included in data provided by personnel in association with delivering the therapy to the target subject.

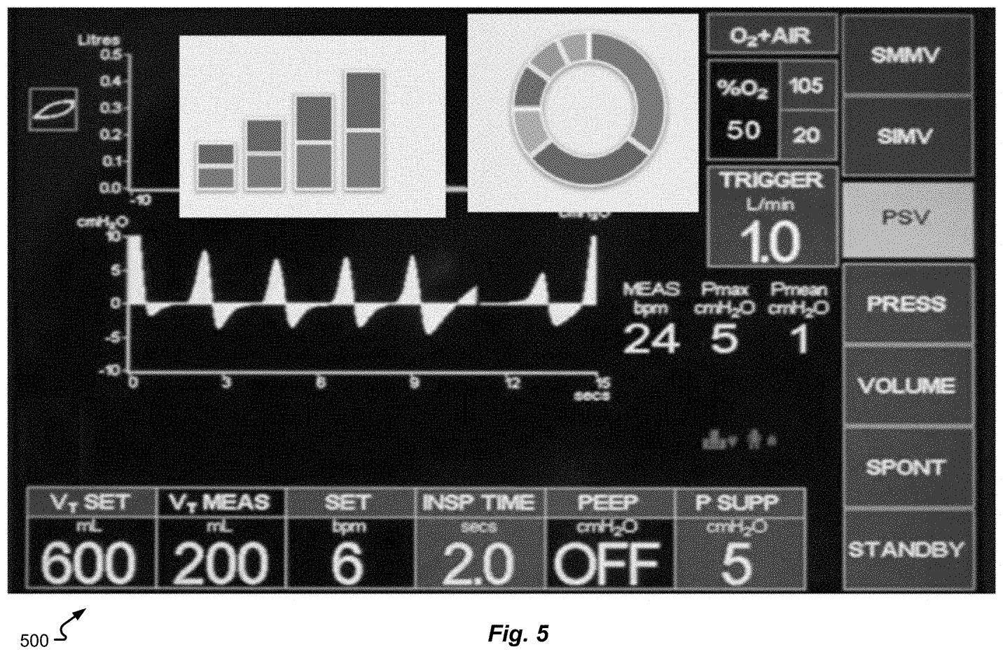

FIG. 5 illustrates an enlarged view 500 of example device settings of a ventilator supported by aspects of the present disclosure.

A number of variations and modifications of the disclosure can be used. It would be possible to provide for some features of the disclosure without providing others.

The phrases “at least one”, “one or more”, “or”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C”, “A, B, and/or C”, and “A, B, or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material”.

The term “computer-readable medium” as used herein refers to any computer-readable storage and/or transmission medium that participate in providing instructions to a processor for execution. Such a computer-readable medium can be tangible, non-transitory, and non-transient and take many forms, including but not limited to, non-volatile media, volatile media, and transmission media and includes without limitation random access memory (“RAM”), read only memory (“ROM”), and the like. Non-volatile media includes, for example, NVRAM, or magnetic or optical disks. Volatile media includes dynamic memory, such as main memory. Common forms of computer-readable media include, for example, a floppy disk (including without limitation a Bernoulli cartridge, ZIP drive, and JAZ drive), a flexible disk, hard disk, magnetic tape or cassettes, or any other magnetic medium, magneto-optical medium, a digital video disk (such as CD-ROM), any other optical medium, punch cards, paper tape, any other physical medium with patterns of holes, a RAM, a PROM, and EPROM, a FLASH-EPROM, a solid state medium like a memory card, any other memory chip or cartridge, a carrier wave as described hereinafter, or any other medium from which a computer can read. A digital file attachment to e-mail or other self-contained information archive or set of archives is considered a distribution medium equivalent to a tangible storage medium. When the computer-readable media is configured as a database, it is to be understood that the database may be any type of database, such as relational, hierarchical, object-oriented, and/or the like. Accordingly, the disclosure is considered to include a tangible storage medium or distribution medium and prior art-recognized equivalents and successor media, in which the software implementations of the present disclosure are stored. Computer-readable storage medium commonly excludes transient storage media, particularly electrical, magnetic, electromagnetic, optical, magneto-optical signals.

Aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Any combination of one or more computer readable medium(s) may be utilized. The computer readable medium may be a computer readable signal medium or a computer readable storage medium.

A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device. Program code embodied on a computer readable medium may be transmitted using any appropriate medium, including but not limited to wireless, wireline, optical fiber cable, RF, etc., or any suitable combination of the foregoing.

The term “control-flow graph (CFG)” is a representation, using graph notation, of all paths that might be traversed through a program during its execution. In a control-flow graph each node in the graph represents a basic block, i.e. a straight-line piece of code without any jumps or jump targets; jump targets start a block, and jumps end a block. Directed edges are used to represent jumps in the control flow. There are, in most presentations, two specially designated blocks: the entry block, through which control enters into the flow graph, and the exit block, through which all control flow leaves. The CFG can thus be obtained, at least conceptually, by starting from the program's (full) flow graph—i.e. the graph in which every node represents an individual instruction—and performing an edge contraction for every edge that falsifies the predicate above, i.e. contracting every edge whose source has a single exit and whose destination has a single entry. This contraction-based algorithm is of no practical importance, except as a visualization aid for understanding the CFG construction, because the CFG can be more efficiently constructed directly from the program by scanning it for basic blocks.

The term “deep learning” refers to machine learning methods based on artificial neural networks. Learning can be supervised, semi-supervised, or unsupervised. Deep learning architectures include deep neural networks, deep belief networks, recurrent neural networks, and convolutional neural networks.

The terms “determine”, “calculate” and “compute,” and variations thereof, as used herein, are used interchangeably and include any type of methodology, process, mathematical operation or technique.

The term “imitation learning” techniques aim to mimic human behavior in a given task. An agent (a learning machine) is trained to perform a task from demonstrations by learning a mapping between observations and actions. For example, imitation learning can directly generalize the expert strategy, observed in the demonstrations, to unvisited states and therefore can be similar to classification, when there is a finite set of possible decisions). In some implementations, an agent follows a “teacher agent” through a policy under the assumption that the teacher agent is maximizing its perceived rewards under the policy (e.g., a reward function). This is assumed to be optimal and may be given by another agent, such as a human expert, to determine the policy. Stated differently, imitation learning is an attempt by a computation device to recover the reward function. Different methods can be used to learn a policy from a demonstration, such as direct imitation, classification, regression, hierarchical models, indirect learning, reinforcement learning, optimization, transfer learning, active learning, apprenticeship learning, active learning, and structured predictions. The paradigm of learning by imitation is gaining popularity because it can facilitate teaching complex tasks with minimal expert knowledge of the tasks. Generic imitation learning methods can potentially reduce the problem of teaching a task to that of providing demonstrations, without the need for explicit programming or designing reward functions specific to the task. Modern sensors can collect and transmit high volumes of data rapidly, and processors with high computational power can allow fast processing that maps the sensory data to actions in a timely manner.

The term “machine learning algorithms” refers to algorithms that effectively perform a specific task without using explicit instructions, relying on patterns and inference instead. It is normally seen as a subset of artificial intelligence. Machine learning algorithms build a mathematical model based on sample data, known as “training data”, to make predictions or decisions without being explicitly programmed to perform the task. Machine learning algorithms include, for example supervised and semi-supervised learning algorithms, unsupervised learning algorithms, and reinforcement learning algorithms.

The term “means” as used herein shall be given its broadest possible interpretation in accordance with 35 U.S.C., Section(s) 112(f) and/or 112, Paragraph 6. Accordingly, a claim incorporating the term “means” shall cover all structures, materials, or acts set forth herein, and all of the equivalents thereof. Further, the structures, materials or acts and the equivalents thereof shall include all those described in the summary, brief description of the drawings, detailed description, abstract, and claims themselves.

The term “module” as used herein refers to any known or later developed hardware, software, firmware, artificial intelligence, fuzzy logic, or combination of hardware and software that is capable of performing the functionality associated with that element.

The preceding is a simplified summary of the disclosure to provide an understanding of some aspects of the disclosure. This summary is neither an extensive nor exhaustive overview of the disclosure and its various aspects, embodiments, and/or configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and/or configurations of the disclosure are possible utilizing, alone or in combination, one or more of the features set forth above or described in detail below. Also, while the disclosure is presented in terms of exemplary embodiments, it should be appreciated that individual aspects of the disclosure can be separately claimed.

The term “reinforcement learning” refers to an area of machine learning concerned with how software agents ought to take actions in an environment so as to maximize some notion of cumulative reward. Reinforcement learning is considered as one of three machine learning paradigms, alongside supervised learning and unsupervised learning. It differs from supervised learning in that labelled input/output pairs need not be presented, and sub-optimal actions need not be explicitly corrected. Instead the focus is finding a balance between exploration (of uncharted territory) and exploitation (of current knowledge). The environment is typically formulated as a Markov decision process (MDP), as many reinforcement learning algorithms for this context utilize dynamic programming techniques. The main difference between the classical dynamic programming methods and reinforcement learning algorithms is that the latter do not assume knowledge of an exact mathematical model of the MDP and they target large MDPs where exact methods become infeasible.

Examples of the processors as described herein may include, but are not limited to, at least one of Qualcomm® Snapdragon® 800 and 801, Qualcomm® Snapdragon® 610 and 615 with 4G LTE Integration and 64-bit computing, Apple® A7 processor with 64-bit architecture, Apple® M7 motion coprocessors, Samsung® Exynos® series, the Intel® Core™ family of processors, the Intel® Xeon® family of processors, the Intel® Atom™ family of processors, the Intel Itanium® family of processors, Intel® Core® i5-4670K and i7-4770K 22 nm Haswell, Intel® Core® i5-3570K 22 nm Ivy Bridge, the AMD® FX™ family of processors, AMD® FX-4300, FX-6300, and FX-8350 32 nm Vishera, AMD® Kaveri processors, Texas Instruments® Jacinto C6000™ automotive infotainment processors, Texas Instruments® OMAP™ automotive-grade mobile processors, ARM® Cortex™-M processors, ARM® Cortex-A and ARM926EJ-S™ processors, other industry-equivalent processors, and may perform computational functions using any known or future-developed standard, instruction set, libraries, and/or architecture.

Any of the steps, functions, and operations discussed herein can be performed continuously and automatically.

The exemplary systems and methods of this disclosure have been described in relation to artificial intelligence. However, to avoid unnecessarily obscuring the present disclosure, the preceding description omits a number of known structures and devices. This omission is not to be construed as a limitation of the scopes of the claims. Specific details are set forth to provide an understanding of the present disclosure. It should however be appreciated that the present disclosure may be practiced in a variety of ways beyond the specific detail set forth herein.

Furthermore, while the exemplary aspects, embodiments, and/or configurations illustrated herein show the various components of the system collocated, certain components of the system can be located remotely, at distant portions of a distributed network, such as a LAN and/or the Internet, or within a dedicated system. Thus, it should be appreciated, that the components of the system can be combined in to one or more devices, such as a server, or collocated on a particular node of a distributed network, such as an analog and/or digital telecommunications network, a packet-switch network, or a circuit-switched network. It will be appreciated from the preceding description, and for reasons of computational efficiency, that the components of the system can be arranged at any location within a distributed network of components without affecting the operation of the system. For example, the various components can be located in a switch such as a PBX and media server, gateway, in one or more communications devices, at one or more users' premises, or some combination thereof. Similarly, one or more functional portions of the system could be distributed between a telecommunications device(s) and an associated computing device.

Furthermore, it should be appreciated that the various links connecting the elements can be wired or wireless links, or any combination thereof, or any other known or later developed element(s) that is capable of supplying and/or communicating data to and from the connected elements. These wired or wireless links can also be secure links and may be capable of communicating encrypted information. Transmission media used as links, for example, can be any suitable carrier for electrical signals, including coaxial cables, copper wire and fiber optics, and may take the form of acoustic or light waves, such as those generated during radio-wave and infra-red data communications.

Also, while the flowcharts have been discussed and illustrated in relation to a particular sequence of events, it should be appreciated that changes, additions, and omissions to this sequence can occur without materially affecting the operation of the disclosed embodiments, configuration, and aspects.

A number of variations and modifications of the disclosure can be used. It would be possible to provide for some features of the disclosure without providing others.

In yet another embodiment, the systems and methods of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein can be used to implement the various aspects of this disclosure. Exemplary hardware that can be used for the disclosed embodiments, configurations and aspects includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

In yet another embodiment, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or VLSI design. Whether software or hardware is used to implement the systems in accordance with this disclosure is dependent on the speed and/or efficiency requirements of the system, the particular function, and the particular software or hardware systems or microprocessor or microcomputer systems being utilized.

In yet another embodiment, the disclosed methods may be partially implemented in software that can be stored on a storage medium, executed on programmed general-purpose computer with the cooperation of a controller and memory, a special purpose computer, a microprocessor, or the like. In these instances, the systems and methods of this disclosure can be implemented as program embedded on personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

Although the present disclosure describes components and functions implemented in the aspects, embodiments, and/or configurations with reference to particular standards and protocols, the aspects, embodiments, and/or configurations are not limited to such standards and protocols. Other similar standards and protocols not mentioned herein are in existence and are considered to be included in the present disclosure. Moreover, the standards and protocols mentioned herein and other similar standards and protocols not mentioned herein are periodically superseded by faster or more effective equivalents having essentially the same functions. Such replacement standards and protocols having the same functions are considered equivalents included in the present disclosure.

The present disclosure, in various aspects, embodiments, and/or configurations, includes components, methods, processes, systems and/or apparatus substantially as depicted and described herein, including various aspects, embodiments, configurations embodiments, subcombinations, and/or subsets thereof. Those of skill in the art will understand how to make and use the disclosed aspects, embodiments, and/or configurations after understanding the present disclosure. The present disclosure, in various aspects, embodiments, and/or configurations, includes providing devices and processes in the absence of items not depicted and/or described herein or in various aspects, embodiments, and/or configurations hereof, including in the absence of such items as may have been used in previous devices or processes, e.g., for improving performance, achieving ease and\or reducing cost of implementation.

The foregoing discussion has been presented for purposes of illustration and description. The foregoing is not intended to limit the disclosure to the form or forms disclosed herein. In the foregoing Detailed Description for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the description has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A system comprising:
a processor; and
a memory storing instructions thereon that, when executed by the processor, cause the processor to:
collect a set of data associated with a target subject;
select a protocol based on the set of data, the protocol comprising one or more settings associated with delivering therapy to the target subject via a device; and
perform a control measure in response to selecting the protocol, wherein performing the control measure comprises delivering the therapy to the target subject, via the device, based on the protocol.

2. The system of claim 1, wherein:
the set of data comprises at least one of:
physiological information associated with the target subject; and
demographics information associated with the target subject; and
selecting the protocol is based on at least one of the physiological information and the demographics information.

3. The system of claim 1, wherein:
the set of data comprises sedation information associated with the target subject, the sedation information comprising at least one of:
one or more sedation settings associated with sedating the target subject, the one or more sedation settings comprising a sedation type and a sedation dosage; and
a degree of consciousness of the target subject; and
selecting the protocol is based on the sedation information.

4. The system of claim 1, wherein:
the set of data comprises intubation information associated with a set of intubations and the target subject, the intubation information comprising at least one of:
a quantity of the set of intubations with respect to a temporal instance or a temporal period; and
a temporal duration associated with an existing intubation of the set of intubations; and
selecting the protocol is based on the intubation information.

5. The system of claim 1, wherein the instructions are further executable by the processor to:
assign a classification to the target subject based on the set of data, wherein selecting the protocol is based on the classification.

6. The system of claim 1, wherein performing the control measure comprises:
transmitting the one or more settings to the device, a communication device associated with one or more personnel, or both.

7. The system of claim 1, wherein the instructions are further executable by the processor to:
collect a second set of data associated with the target subject in response to performing the control measure;
perform a second control measure in response to processing the second set of data, wherein performing the second control measure comprises:
adjusting or maintaining the one or more settings based on the second set of data; and
delivering therapy to the target subject, via the device, based on adjusting or maintaining the one or more settings.

8. The system of claim 7, wherein the instructions are further executable by the processor to:
compare at least a portion of the second set of data to a set of target criteria, the set of target criteria comprising at least one of:
a target physiological parameter; and
a target treatment outcome; and
adjusting or maintaining the one or more settings based on a result of the comparing.

9. The system of claim 1, wherein:
the protocol comprises a baseline configuration associated with delivering the therapy to the target subject,
wherein the baseline configuration comprises:
the one or more settings; and
respective weighting factors corresponding to the one or more settings.

10. The system of claim 1, wherein:
the device comprises a ventilator; and
the one or more settings comprise one or more device settings associated with the ventilator.

11. The system of claim 1, wherein the one or more settings comprise:
a recommendation to intubate or extubate the target subject; and
temporal information associated with the recommendation.

12. The system of claim 1, wherein the instructions are further executable by the processor to:
provide at least a portion of the set of data to a machine learning model; and
receive an output from the machine learning model in response to the machine learning model processing at least the portion of the set of data, the output comprising at least one of:
an indication of a classification to the target subject;
an indication of the protocol; and
an indication of the one or more settings.

13. The system of claim 12, wherein processing at least the portion of the set of data by the machine learning model comprises:
generating predicted physiological information associated with the target subject based on at least the portion of the set of data; and
comparing the predicted physiological information to target physiological information,
wherein the output from the machine learning model is based on a result of the comparing.

14. The system of claim 12, wherein:
the machine learning model performs one or more iterations of a control system loop, the control system loop comprising:
providing the output;
collecting an additional set of data, the additional data comprising physiological information associated with the target subject;
generating additional predicted physiological information;
comparing the additional predicted physiological information to the target physiological information; and
providing an additional output, the additional output comprising one or more additional settings associated with delivering the therapy to the target subject via the device.

15. The system of claim 1, wherein the machine learning model is a software machine learning model.

16. The system of claim 1, wherein the instructions are further executable by the processor to:
train and validate the machine learning model based on a comparison of the one or more settings to historical data associated with delivering the therapy to a set of subjects,
wherein the historical data comprises a set of previously applied settings associated with delivering the therapy to the set of subjects.

17. The system of claim 1, wherein the instructions are further executable by the processor to:
train and validate the machine learning model based on a comparison of the one or more settings to a set of proposed settings associated with delivering the therapy to the target subject,
wherein the set of proposed settings is included in data provided by personnel in association with delivering the therapy to the target subject.

18. A system comprising:
a therapy device;
a processor; and
a memory storing instructions thereon that, when executed by the processor, cause the processor to:
collect a set of data associated with a target subject;
select a protocol based on the set of data, the protocol comprising one or more settings associated with delivering therapy to the target subject via the therapy device; and
perform a control measure in response to selecting the protocol, wherein performing the control measure comprises delivering the therapy to the target subject, via the therapy device, based on the protocol.

19. The system of claim 18, wherein:
the set of data comprises at least one of:
physiological information associated with the target subject; and
demographics information associated with the target subject; and
selecting the protocol is based on at least one of the physiological information and the demographics information.

20. A method comprising:
collecting a set of data associated with a target subject;
selecting a protocol in response to collecting the set of data, the protocol comprising one or more settings associated with delivering therapy to the target subject via a device; and
performing a control measure in response to selecting the protocol, wherein performing the control measure comprises delivering the therapy to the target subject, via the device, based on the protocol.
